(19) United States
(12) Patent Application Publication
(10) Pub. No.: US 2015/0093543 A1
(43) Pub. Date: Apr. 2, 2015

(54) BLOOD COMPATIBLE SURFACES

(71) Applicants: University of Massachusetts, Boston, MA (US); Teijin Limitied, Osaka (JP)

(72) Inventors: Takashi Kushida, Tokyo (JP); Vincent M. Rotello, Amherst, MA (US)

(21) Appl. No.: 14/501,433

(22) Filed: Sep. 30, 2014

Related U.S. Application Data
(60) Provisional application No. 61/884,956, filed on Sep. 30, 2013.

(51) Int. Cl. A61L 33/00; A61L 33/02

(52) U.S. Cl. CPC A61L 33/0094; A61L 33/027; A61L 2400/12; A61L 2420/02
USPC 428/143; 428/141; 134/42; 427/2.1; 264/293; 128/898

(57) ABSTRACT

The disclosure features blood compatible articles and methods of making the articles. The methods include providing a substrate and forming a rough surface on the substrate. The rough surface includes a plurality of three-dimensionally curved features each having a radius of curvature of less than about 50 nm. The surface includes a sufficient concentration of features per unit area to limit blood coagulation activity on the substrate and to limit the number of platelets that adhere to the surface when the substrate is exposed to blood.

### CLAIM OF PRIORITY

This application claims priority under 35 USC §119(e) to U.S. Patent Application Ser. No. 61/884,956, filed on Sep. 30, 2013, the entire contents of which are hereby incorporated by reference.

### FIELD OF THE INVENTION

The invention relates to blood compatible surfaces, e.g., blood compatible surfaces formed of nanoparticles.

### BACKGROUND OF THE INVENTION

Medical devices, such as hemodialysis membranes, artificial blood vessels, heart valves, biosensors, vascular stents, and other medical devices are often used for the treatment of various medical conditions. However, when foreign objects such as medical devices come into contact with the blood of a patient, a series of adverse biological reactions can be triggered, including thrombosis, inflammation, and fibrosis. These reactions can be harmful to the patient and can cause failure of the implanted medical device.

To limit these adverse biological reactions, blood compatible materials can be used for such medical devices. Blood compatible materials limit the activation of the blood coagulation system and reduce or prevent platelet adhesion to the material. Surface treatments can be applied to medical devices to improve the blood compatibility of the devices. For instance, self-assembled monolayers, polyethylene oxide, heparin, zwitterionic polymers, and inorganic coatings such as diamond can be applied to the surface of medical devices.

### SUMMARY OF THE INVENTION

The invention is based, at least in part, on the discovery that high curvature surfaces, such as coatings formed of nanoparticles having a diameter less than about 100 nm, exhibit blood compatible properties. For instance, high curvature blood compatible surfaces, such as coatings formed of nanoparticles, limit the intrinsic coagulation activity of blood in the vicinity of the blood compatible surface. Furthermore, high curvature blood compatible surfaces limit the adsorption of platelets onto the surface. In some cases, when medical devices come into contact with the blood of a patient, adverse biological reactions, such as blood coagulation on surfaces of the medical device and platelet adhesion to the device, can occur. By covering medical devices with high curvature blood compatible surfaces, such adverse biological reactions can be mitigated.

In a general aspect, methods of making blood compatible articles as described herein include providing a substrate; and forming a rough surface on the substrate. The rough surface includes a plurality of three-dimensionally curved features each having a radius of curvature of less than about 50 nm, e.g., 5, 10, 15, 20, 25, 30, 35, 40, or 45 nm. The surface includes a sufficient concentration of features per unit area to limit blood coagulation activity on the substrate and to limit the number of platelets that adhere to the surface when the substrate is exposed to blood.

Embodiments can include one or more of the following features. The three-dimensionally curved features can be substantially hemispherical. The rough surface can include a coating on the substrate, and the coating can include the features. The features can include nanoparticles and the fill rate of the nanoparticles in the coating can be at least about 50%, e.g., at least about 60% or at least about 70%. The features can be nanoparticles having a diameter of less than about 100 nm. The diameter of the nanoparticles can be less than about 85 nm, e.g., between about 12 nm and about 85 nm, e.g., 15, 20, 25, 30, 35, 40, 45, 50, 55, 60, 65, 70, 75, or 80 nm. The diameter of the nanoparticles can be the average particle size of the nanoparticles as determined by a dynamic light scattering method. For example, the nanoparticles can include one or more of ceramic nanoparticles, metal nanoparticles, metal oxide nanoparticles, and polymer nanoparticles.

Forming the coatings can include spin coating the nanoparticles onto the surface of the substrate, e.g., of a medical device or blood container. Spin coating the nanoparticles can include spin coating a suspension of nanoparticles in an alcohol, such as ethanol. Forming the coating can include annealing the spin coated nanoparticles, e.g., once they are adhered to the substrate. Forming the coating can include one or more of dip coating the nanoparticles onto the surface of the substrate, spray coating the nanoparticles onto the surface, precipitating the nanoparticles onto the surface, and depositing the nanoparticles by flame spray pyrolysis. Forming the coating can include forming the features by nano-imprinting on the substrate. The features also can be formed of a biocompatible material.

The substrate can be a medical device or part of a medical device, such as an implantable medical device, e.g., a surgical device, an implantable device, a blood pump, a blood container, or a conduit for blood transport. The medical device can be configured for exposure to blood outside of the body of a patient or within a patient. The method can be carried out in vivo (e,g. within a patient) or ex vivo (e.g., outside of the body of a patient).

An RMS (root mean square) roughness of the surface can be less than about 10 nm, e.g., less than about 5 nm, e.g., between 0.5 nm and 10 nm.

In another general aspect, blood compatible articles as described herein include a substrate having a rough surface. The rough surface includes a plurality of three-dimensionally curved features each having a radius of curvature of less than about 50 nm, e.g., 5, 10, 15, 20, 25, 30, 35, 40, or 45 nm. The surface includes a sufficient concentration of features per unit area to limit blood coagulation activity on the substrate and to limit the number of platelets that adhere to the surface when the substrate is exposed to blood.

Embodiments can include one or more of the following features. The features can be substantially hemispherical. The rough surface can include a coating on the substrate, wherein the coating comprises the features. The features can include nanoparticles and a fill rate of the nanoparticles in the coating can be at least about 50%, e.g., at least about 60% or at least about 70%. The features can be nanoparticles having a diameter of less than about 100 nm, e.g., less than about 85 nm, e.g., between about 12 nm and about 85 nm, e.g., 15, 20, 25, 30, 35, 40, 45, 50, 55, 60, 65, 70, 75, or 80 nm. The diameter of the nanoparticles can be the average particle size of the nanoparticles as determined by a dynamic light scattering method.

The coating can be or include a ceramic, metal, metal oxide, or polymer material, or can be or include mixtures of one or more of these materials. The coating can be non-toxic and/or biocompatible.

The substrate can be a medical device or part of a medical device.

The concentration of the features can limit the adsorption onto the substrate of one or more proteins associated with coagulation. For example, the concentration of the features can limit the adsorption of Factor XII onto the substrate. For example, the limited coagulation activity can inhibit formation of a fibrin clot at the surface of the substrate. The concentration of the features can limit the activation of platelets adsorbed on the substrate. An RMS roughness of the surface can be less than about 10 nm, e.g., less than about 5 nm, e.g., between 0.5 nm and 10 nm.

The term “blood compatible” refers to the ability of a material to limit the activation of the blood coagulation system in the vicinity of the material and to prevent platelet adhesion to the material.

The blood compatible surfaces described herein have a number of advantages. For instance, medical devices that come into contact with a patient's blood can be treated with or manufactured with blood compatible surfaces to reduce adverse biological reactions associated with the use of such medical devices. The blood compatible surface can act as a barrier between the medical device and blood, thus allowing a wider range of materials to be used for the medical device itself. For instance, medical devices that exhibit or are treated with blood compatible coatings or surfaces can be formed of materials that are inexpensive, readily available, or easy to process, even if those materials are not biocompatible without the blood compatible coatings or surfaces.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a diagram of a blood compatible coating formed of nanoparticles.

FIG. 1B is a diagram of a high curvature blood compatible coating.

FIG. 2 is a flow chart of a coagulation cascade.

FIG. 3 is a diagram of an implanted medical device.

FIGS. 4A-4D are atomic force microscopy images of blood compatible coatings of nanoparticles of different average diameters.

FIGS. 5A-5F are scanning electron microscopy images of blood compatible coatings of nanoparticles.

FIGS. 6A and 6B are a GISAXS (Grazing-incidence small-angle X-ray scattering) image and spectrum, respectively, for a blood compatible coating of 50 nm nanoparticles, respectively.

FIGS. 6C and 6D are a GISAXS image and spectrum, respectively, for a blood compatible coating of 12 nm nanoparticles.

FIGS. 7A-7D are plots of the time dependence of the intrinsic coagulation activity for nanoparticles in suspension as a function of particle size and concentration.

FIG. 8 is a plot of the intrinsic coagulation activity for nanoparticles in suspension as a function of nanoparticle size.

FIG. 9 is a plot of the intrinsic coagulation activity for nanoparticles in suspension as a function of nanoparticle size and concentration.

FIGS. 10A-10C are plots of the intrinsic coagulation activity on blood compatible coatings of nanoparticles of different sizes after incubation for 90 minutes, 180 minutes, and 300 minutes, respectively.

FIGS. 11A-11F are optical microscopy images of platelets adhered to blood compatible coatings of nanoparticles of different sizes.

FIG. 12 is a plot of the number of platelets adhered to blood compatible coatings of nanoparticles of different sizes.

### DETAILED DESCRIPTION

As described herein, high curvature surfaces formed of features, such as nanoparticles, with a diameter or widest dimension, e.g., width, of less than about 100 nm, exhibit blood compatible properties. For instance, high curvature blood compatible surfaces can limit the intrinsic coagulation activity of blood in the vicinity of the surfaces, thus preventing the formation of fibrin clots at the coatings or surfaces. Furthermore, high curvature blood compatible surfaces can limit the adhesion of platelets, thus preventing the formation of platelet plugs and/or clots at the surfaces.

In some cases, when medical devices come into contact with the blood of a patient, adverse biological reactions can occur, such as blood coagulation and/or platelet accumulation. By coating medical devices with high curvature blood compatible surfaces, or by forming such devices with such surfaces, these adverse biological reactions can be mitigated.

### Structure and Fabrication of Blood Compatible Coatings

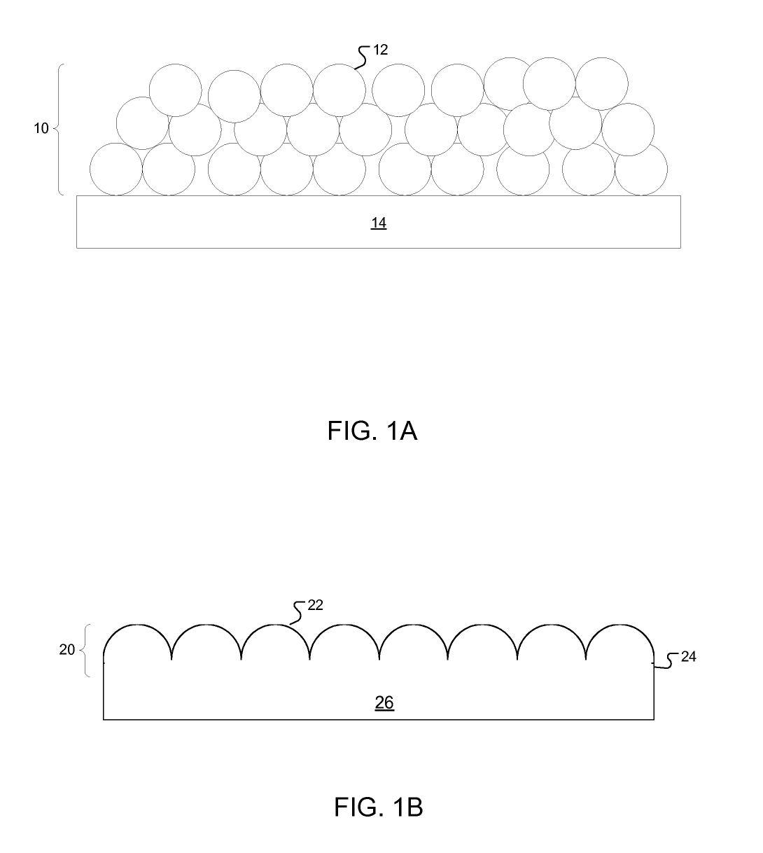

Referring to FIGS. 1A and 1B, in one embodiment, a blood compatible coating 10 on a substrate 14 includes features with high curvature (i.e., materials with a small radius of curvature), such as three-dimensionally curved features that are approximately hemispherical. For instance, as shown in FIG. 1A, the blood compatible coating 10 can be formed of nanoparticles 12 that are disposed on the substrate 14. In different examples, the nanoparticles 12 can have a diameter of less than about 100 nm, e.g., less than about 85 nm. For instance, in various examples, the nanoparticles 12 can have a diameter of about 4, 5, 6, 7, 8, 9, 10, 12, 15, 20, 25, 30, 35, 40, 45, 50, 60, 70, 80, 85, 90, or 95 nm. In some examples, the root mean square (RMS) roughness of the blood compatible coating 10 is less than about 10 nm, or between about 0.5 nm and about 10 nm.

The nanoparticles 12 can be formed of a biocompatible material. In some examples, the nanoparticles 12 can be formed of a ceramic material, such as silica (SiO₂), titanium dioxide (TiO₂), zirconia (ZrO₂), zinc oxide (ZnO), aluminum oxide (Al₂O₃), iron oxide (Fe₃O₄), or another ceramic, such as a biocompatible ceramic. In some cases, the nanoparticles 12 can be fabricated, e.g., by solution-based synthesis procedures. In some cases, the nanoparticles 12 can be grown on the surface of the substrate 14, e.g., in a vapor-phase deposition process, a flame spray pyrolysis approach, a chemical precipitation approach, or another approach to growing nanoparticles. In some examples, the nanoparticles 12 can be formed of polymers, such as biocompatible polymers. For example, polystyrene, polyethylene, polypropylene, polycaprolactone, polylactic acid, polyglycolide, poly(lactide-co-glycolide), polyacrylate derivatives, cellulose and chitin can be used to form the nanoparticles 12.

In some examples, the nanoparticles 12 in the blood compatible coating 10 can interact with each other via chemical interactions such as van der Waals interactions, electrostatic interactions, hydrogen bonds, or another type of chemical interaction. In some examples, the nanoparticles 12 can be functionalized to bind together to form a cross-linked network of nanoparticles. For example, the nanoparticles 12 can be functionalized with ligands having end groups that can bind to other nanoparticles 12 or to the end groups of other ligands.

The substrate 14 can be any material that provides a desired function or property. For instance, the substrate 14 can be a medical device to be implanted into the body of a patient or a medical device that handles blood outside the body. For instance, if the coating 10 is applied to a coronary stent, the substrate 14 can be the material of the coronary stent. In some examples, the substrate 14 can be functionalized to chemically bind the nanoparticles 12 to the substrate 14. For instance, the substrate 14 can be functionalized with siloxane-terminated molecules that can covalently bond to silica nanoparticles.

In some examples, the blood compatible coating 10 of nanoparticles 12 can be formed by spin coating a dispersion of nanoparticles 12 in alcohol, such as ethanol, onto the substrate 14. For instance, a dispersion of nanoparticles 12 in ethanol can be spin-coated onto the substrate 14. In some examples, the coating 10 of nanoparticles 12 can be formed by dip coating the substrate 14 into a dispersion of nanoparticles 12 in alcohol, such as ethanol. In some examples, the coating 10 can be annealed following spin or dip coating, e.g., to promote chemical interaction (e.g., van der Waals binding) between nanoparticles 12 in the coating 10.

The thickness of the coating 10 of nanoparticles 12 can be less than 1 mm, e.g., less than about 500 nm, less than about 250 nm, less than about 100 nm, less than about 50 nm, less than about 25 nm, or less than about 10 nm. For instance, the coating 10 can be formed of about two layers of nanoparticles 12, and thus the thickness of the coating 10 can be about twice the diameter of the nanoparticles 12. A coating 10 formed of fewer than two layers of nanoparticles 12 can have exposed areas of substrate 14, which reduces the effectiveness of the blood compatible coating 10. If the coating 10 is too thick (e.g., thicker than about 1 mm), the coating 10 can easily crack.

The thickness of the coating 10 of nanoparticles 12 can be controlled by varying process parameters, such as the concentration of nanoparticles 12 in ethanol, the rotation speed of the spin coating, the acceleration of the spin coater, the number of repetitions of spin coating, and other parameters. For instance, the weight percent concentration of nanoparticles in ethanol can range from about 0.05 wt. % to about 10 wt. %, e.g., about 1.3 wt. %, about 3.0 wt. %, or about 4.0 wt. %. The rotation speed of the spin coating can range from about 100 rpm to about 10000 rpm, e.g., about 1000 rpm, about 2000 rpm, or about 3000 rpm. The acceleration of the spin coater can range from about 400 rpm/s to 4000 rpm/s.

The nanoparticles 12 in the coating 10 are densely packed. For instance, the fill rate (i.e., the percentage of space in the coating 10 that is occupied by nanoparticles 12) in the coating 10 is at least about 50%, e.g., at least about 60%, 65%, or 70%.

In some examples, other approaches to forming the blood compatible coating 10 of nanoparticles 12 can be used. In some cases, nanoparticles 12 can be spray-coated onto the substrate 14. In some cases, nanoparticles 12 can be grown directly on the substrate 14, e.g., in a vapor-phase deposition process. In some cases, nanoparticles 12 can be disposed on the surface by a Langmuir-Blodgett approach to forming coatings of nanoparticles, a layer-by-layer deposition of nanoparticles from a dispersion in a solvent, a spray pyrolysis approach, a chemical precipitation approach, or another approach. In the Langmuir-Blodgett method, a dispersion of nanoparticles in an organic solvent with appropriate surfactant is spread on a water surface to make a film of nanoparticles on the water surface. The film of nanoparticles is transferred to a solid surface from the water surface. In the layer-by-layer method, a positively charged nanoparticle dispersion and a negatively charged nanoparticle dispersion are prepared. When a base substrate is positively charged, the substrate is dipped into the negatively charged particle dispersion and then dipped into the positively charged particle dispersion. Nanoparticles are deposited on the base substrate by electric force. In the spray pyrolysis approach, a precursor solution is sprayed onto a substrate with heat under appropriate conditions. In chemical precipitation, a substrate is placed at the bottom of a precursor solution. Nanoparticles are created from the precursor solution by a reaction such as a redox reaction and precipitated onto the substrate directly.

Referring to FIG. 1B, in some embodiments, blood compatible surfaces 20 can include three-dimensional, highly curved features, such as bumps or peaks and valleys, on a surface 24 of a substrate 26. The features can be approximately hemispherical. For instance, the radius of curvature of the features 22 can be less than 50 nm, e.g., less than about 42.5 nm. For instance, in one example, the features 22 can have a radius of curvature of about 2, 3, 4, 5, 6, 7, 10, 12, 15, 20, 25, 30, 35, 40, 45, or 50 nm.

The surfaces 20 can be formed by etching (e.g., wet etching or plasma etching) the surface 24 of the substrate 26 to form highly curved nanostructures 22, such as bumps or peaks and valleys, on the surface 24. For instance, surface features having a maximum radius of curvature of less than about 50 nm, or less than about 42.5 nm, can be etched into the surface. In some examples, the RMS roughness of the blood compatible coating 20 is less than about 10 nm, or between about 0.5 nm and about 10 nm.

Wet etch chemistries or plasma etch chemistries can be selected to etch the material of the substrate, e.g., to anisotropically etch the material of the substrate. In some cases, wet chemical etching using chemistries capable of etching silica can be used to form a nanostructured SiO₂ surface. Examples of wet etch chemistries capable of etching silica include, e.g., potassium hydroxide, tetramethylammonium hydroxide, ethylenediamine pyrocatechol, and hydrofluoric acid. In some cases, plasma etching using chemistries capable of etching silica can be used to form a nanostructured SiO₂ surface. Examples of plasma etch chemistries capable of etching silica include, e.g., hydrofluoric acid and buffered oxide etch (which includes ammonium fluoride and hydrofluoric acid). Other wet etch or plasma etch chemistries can be used to etch substrates of other compositions.

In some cases, the etched or machined nanostructured substrate can be applied to a medical device. In some cases, the surface of a medical device can itself be the substrate that is etched or machined such that highly curved nanostructures are formed directly on the medical device.

In some embodiments, blood compatible coatings can be formed by depositing a thin film of a material onto a substrate under deposition conditions that cause the thin film to have a high degree of roughness, such as an RMS roughness of less than about 10 nm, or between about 0.5 nm and about 10 nm. For instance, deposition conditions can be controlled to produce a surface with a roughness that correlates to surface features having a maximum radius of curvature of less than about 50 nm, or less than about 42.5 nm. In some cases, such a thin film can be deposited directly onto the surface of a medical device.

Other fabrication approaches can also be used to form surfaces with highly curved nanostructures. In some examples, substrates can be machined to form nanostructures on the surface of the substrates. In some examples, devices can be formed using a nano-imprinting approach, including forming a nanostructured surface from a mold that includes nanostructured features. For instance, a mold having nanostructured features can be formed using electron beam lithography or other lithography techniques or by forming a mold from a pre-existing nanoparticle layer. A high curvature polymer surface can be fabricated using thermal or photo nanoimprint lithography (NIL) based on a nanostructured mold. In the case of thermal NIL, a thermoplastic polymer film is formed on a substrate, and the mold is pressed into contact with the sample under appropriate pressure. When heated above the glass transition temperature of the polymer, the pattern on the mold is pressed into the softened thermoplastic polymer film. After cooling, the mold is separated from the sample and the pattern remains on the substrate. In the case of photo NIL, a photo-curable polymer liquid resist is applied to the sample substrate and the mold. After the mold and the substrate are pressed together, the resist is cured in UV light and becomes solid. After mold separation, a similar pattern transfer process can be used to transfer the pattern in resist onto the underneath material.

### Uses of Blood Compatible Coatings and Surfaces

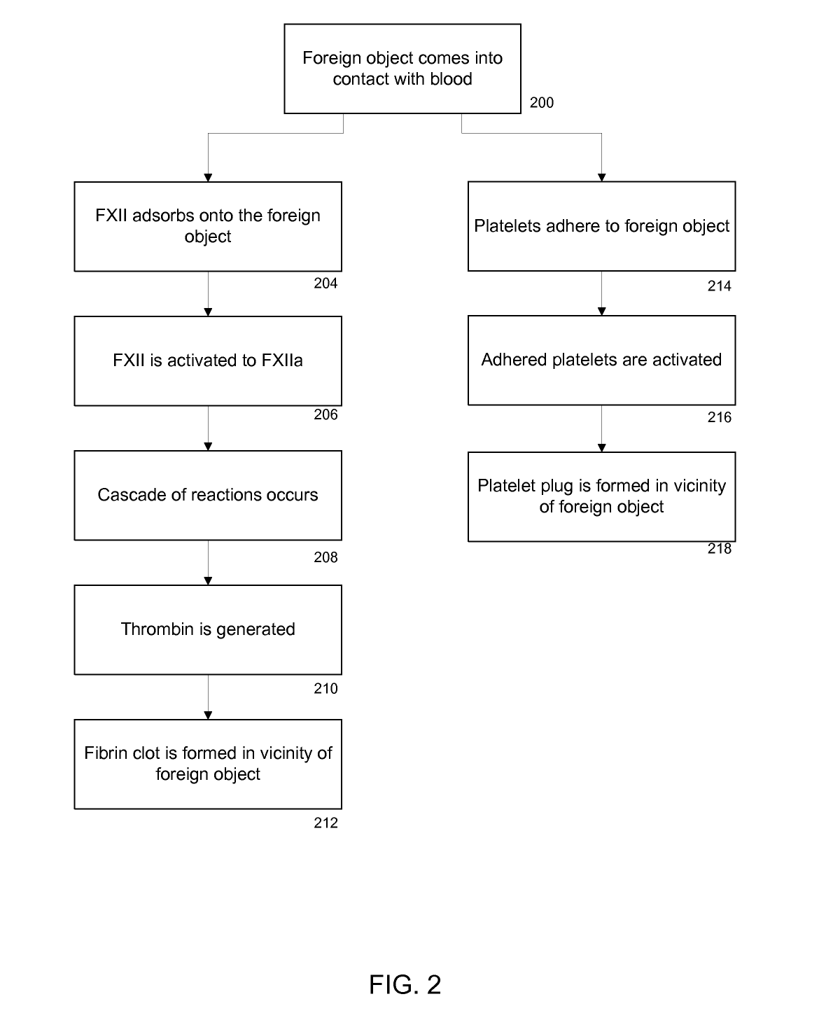

Blood compatible coatings can help to reduce the level of adverse biological reactions that occur when a foreign object comes into contact with blood, either within a subject's body or when a subject's blood passes through a device located outside the body. As shown in FIG. 2, when a foreign object, such as a medical device, comes into contact with blood (200), an intrinsic blood coagulation pathway is activated that involves a cascade of proteolytic reactions (referred to as a coagulation cascade) that results in the formation of a fibrin clot at the foreign object. In particular, Factor XII (referred to as FXII) adsorbs onto the surface of the foreign object (204) and is denatured, thus activating to Factor XIIa (referred to as FXIIa) (206). For instance, hydrophilic or negatively charged surfaces are often highly active materials for FXII denaturation and activation. The intrinsic blood coagulation cascade begins (208) following the activation of FXII into FXIIa that ultimately result in the generation of thrombin (210), a substance that changes fibrinogen to fibrin and causes formation of a fibrin clot (212) in the vicinity of the foreign object. In addition, platelets can adhere to the foreign object (214), e.g., within minutes of the introduction of the foreign object into the blood. The adhered platelets can be activated (216), causing the formation of a platelet plug (218) in the region of the foreign object.

The presence of blood compatible surfaces can reduce the degree of intrinsic coagulation activity in blood exposed to the surfaces. That is, the ability of FXII to adsorb onto a blood compatible coating is less than the ability of FXII to adsorb onto a flat surface of the same composition, and thus the intrinsic coagulation cascade can be weakened in the presence of blood compatible surfaces. The reduced activity of the coagulation cascade due to blood compatible coatings can, in turn, limit the formation of fibrin clots in the vicinity of the coatings.

Furthermore, platelet adhesion can also be reduced in the presence of blood compatible surfaces. That is, the ability of platelets to adhere to a blood compatible surface is less than the ability of platelets to adhere to a flat surface of the same composition, and thus the degree of platelet adhesion can be reduced in the presence of a blood compatible surface. The reduced platelet adhesion to blood compatible surfaces can, in turn, limit the formation of platelet plugs at the surfaces.

Without being bound by theory, it is believed that the limited denaturation of FXII on blood compatible surfaces is due to the high surface curvature of the surfaces (e.g., the high curvature of the nanoparticles or surface features forming the blood compatible surfaces). Furthermore, the limited denaturation of platelets on blood compatible surfaces is also due to the high surface curvature of the surfaces. That is, high curvature surfaces of any composition can limit FXII denaturation and platelet adhesion, provided the concentration (per unit area) of highly curved features on the surface is sufficiently high. Such high curvature surfaces can thus significantly reduce the formation of fibrin clots and platelet plugs. For instance, a high curvature surface, such as a surface formed of SiO₂ nanoparticles, can be blood compatible even if the material of the surface (SiO₂) is not itself a blood compatible material.

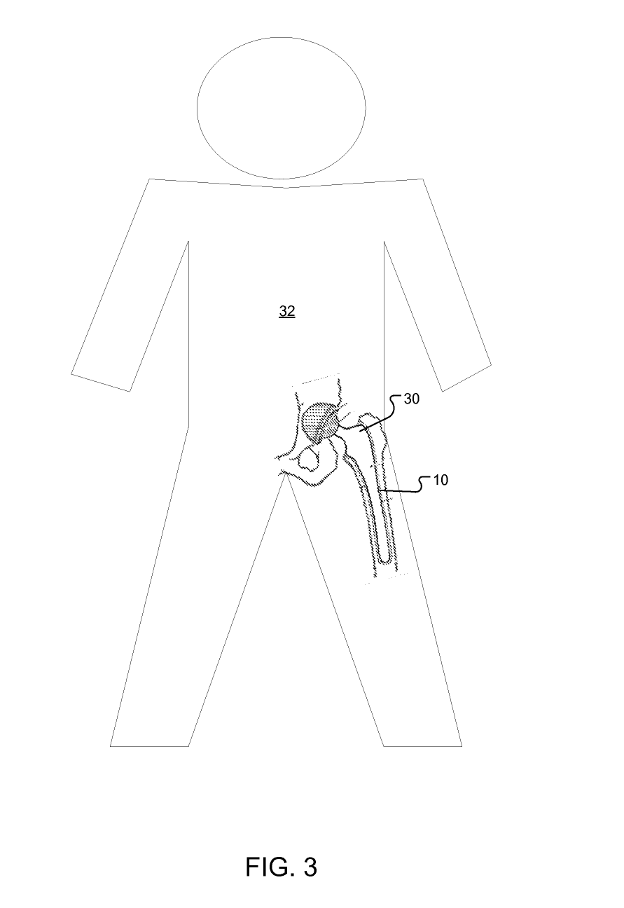

Referring to FIG. 3, in some embodiments, coating 10 can be applied as a coating for an implantable medical device 30. For instance, the implantable medical device 30 can be coated with the coating 10, e.g., by dip coating prior to implantation. In the example of FIG. 3, the implantable medical device 30 is an artificial hip joint; however, the coating 10 can be applied to other implantable medical devices, such as other artificial joints, artificial blood vessels, stents, cochlear implants, pacemakers, implantable defibrillators, bone screws and plates, coronary stents, and other implantable medical devices. When the medical device 30 is implanted into a patient's body 32, the blood compatibility of the coating 10 can reduce the occurrence or severity of adverse biological reactions, such as inflammation and/or formation of blood clots, associated with the implant. Moreover, the coating 10 can act as a barrier between the implanted medical device 30 and the body 32, and thus a wider range of materials can be available to be used for the implanted medical device 30. For instance, the implantable medical device 30 can be formed of a material that is inexpensive, readily available, not blood compatible, and/or not biocompatible.

In some embodiments, the coating 10 can be applied as a coating for medical devices that handle blood outside of the body. For instance, the coating 10 can be applied as a coating within dialysis equipment, blood donation and transfusion equipment, and other medical devices that handle, e.g., contain or transfer, blood outside of the body. The blood compatibility of the coating 10 can reduce the occurrence or severity of blood clots or other adverse reactions in the blood handled by the medical devices. Moreover, the coating 10 can act as a barrier between the medical devices and the blood handled by the devices, and thus a wider range of materials can be available to be used for the medical devices.

### EXAMPLES

The invention is further described in the following examples, which do not limit the scope of the invention described in the claims.

The following examples show an approach to fabricating blood compatible coatings of nanoparticles. The examples further demonstrate intrinsic coagulation activity in suspensions of nanoparticles and on blood compatible coatings of nanoparticles. The examples also demonstrate platelet adhesion on blood compatible coatings of nanoparticles.

### Example 1

### Preparing Blood Compatible Coatings of Nanoparticles

Blood compatible coatings of silica nanoparticles of various sizes were fabricated on Si wafer substrates. 5 mL of silica nanoparticle dispersion in water (various sizes and manufacturers; see Table 1) was added to a vigorously stirred solution of 0.5 mL HCl (aq.) in 44.5 mL ethanol. The concentration of the 12 nm, 22 nm, 50 nm, and 85 nm nanoparticles in water was 40 wt. %; the concentration of the 7 nm nanoparticles in water was 30 wt. %; and the concentration of the 4 nm nanoparticles was 15 wt. %. Each nanoparticle dispersion in ethanol was placed in a 10K molecular weight cutoff dialysis membrane (Fisher Scientific) and dialyzed against ethanol several times.

TABLE 1
Silica nanoparticles used to prepare blood compatible coatings

| | | Nominal | Surface |
|---|---|---|---|
| Commercial | | diameter | area |
| name | Provider | (nm) | (cm²/g) |
| AS | Alfa Aesar ® (Ward Hill, MA) | 4 | 6.5 × 10⁶ |
| Ludox ® SM | Sigma-Aldrich ® (St. Louis, MO) | 7 | 3.45 × 10⁶ |
| Ludox ® HS | Sigma-Aldrich ® | 12 | 2.2 × 10⁶ |
| Ludox ® TM | Sigma-Aldrich ® | 22 | 1.4 × 10⁶ |
| NexSil ™ 85 | Nyacol ® Nano Technologies, Inc. | 50 | 0.55 × 10⁶ |
| | (Ashland, MA) | | |
| NexSil ™ 125 | Nyacol ® Nano Technologies, Inc. | 85 | 0.35 × 10⁶ |

1 cm² pieces of Si wafers were used as substrates. The substrates were sonicated in acetone and ethanol, dried under nitrogen flow, and treated by oxygen plasma for ten minutes. Immediately following the oxygen plasma treatment each dialyzed nanoparticle dispersion in ethanol was spin-coated onto a substrate at 3000 rpm for 160 seconds. The coated substrates were annealed at 100° C. for ten minutes and rinsed with DI water and ethanol.

The refractive index of each nanoparticle coating was measured by ellipsometry to be about 1.31. This refractive index corresponds to a fill rate of about 68% (i.e., nanoparticles occupy about 68% of the space in the coating), indicating that the nanoparticles in the coating are densely packed. Ellipsometry was performed using a Stokes Ellipsometer LSE (Gaertner® Scientific Corporation, Skokie, Ill.).

The thickness of the nanoparticle coatings can be varied by varying parameters such as the concentration of nanoparticles in ethanol and the rotation speed of the spin coating. Ellipsometry measurements of the thickness of each nanoparticle coating were performed at 9 points in each coating to quantify the uniformity of the coating. Coating thicknesses obtained for various nanoparticles sizes, concentrations, and rotation speeds are shown in Table 2. In general, the 9 measurements for each nanoparticle coating were within about 1 nm of each other, indicating a highly uniform thickness.

TABLE 2
Thicknesses of blood compatible coatings of nanoparticles

| Nominal | Concentration | Rotation speed | Thickness |
|---|---|---|---|
| diameter (nm) | (wt. %) | (rpm) | (nm) |
| 85 nm | 4.0 | 3000 | ≈220 |
| 85 nm | 4.0 | 2000 | ≈182 |
| 85 nm | 3.0 | 3000 | ≈100 |
| 85 nm | 3.0 | 2000 | ≈113 |
| 85 nm | 3.0 | 1000 | ≈146 |
| 22 nm | 1.3 | 3000 | ≈69 |
| 22 nm | 1.3 | 2000 | ≈91 |
| 22 nm | 1.3 | 1000 | ≈130 |

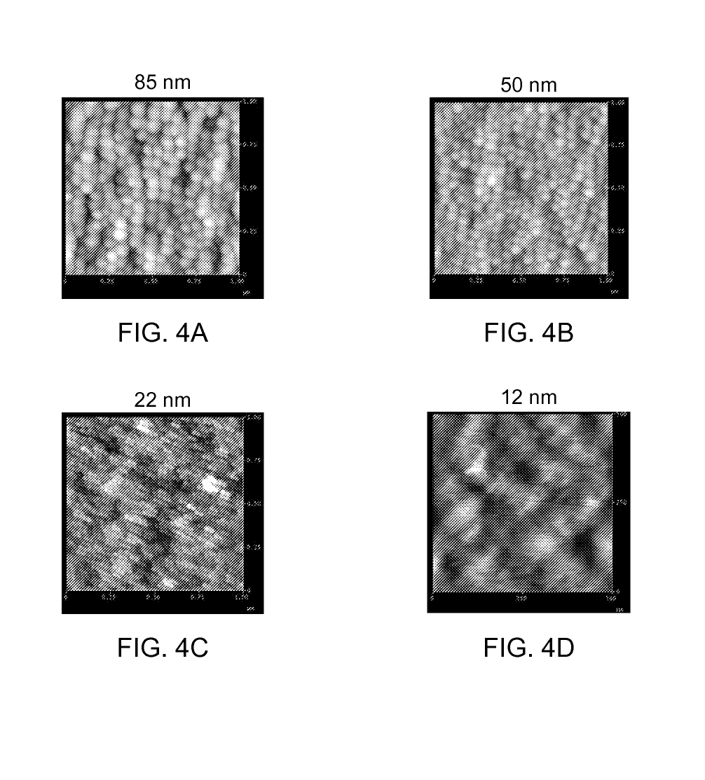

Referring to FIGS. 4A-4D, atomic force microscopy (AFM) images were acquired for blood compatible coatings of nanoparticles with diameters of 85 nm, 50 nm, 22 nm, and 12 nm, respectively. These AFM images show that nanoparticles forming each of the blood compatible coatings are substantially uniform in size.

The root mean square (RMS) roughness of each coating was also determined by AFM. RMS roughness values are listed in Table 3 for a 500 nm×500 nm area of each coating. RMS roughness decreases monotonically with decreasing nanoparticle diameter, suggesting that the surface topology of the blood compatible coating can be controlled by controlling the size of the nanoparticles forming the coating. AFM imaging and measurements were performed in tapping mode using a DI-3000 atomic force microscope (Veeco, Plainview, N.Y.).

TABLE 3
AFM and SEM characterizations of blood
compatible coatings of nanoparticles

| Nominal | | RMS roughness | Diameter (nm) |
|---|---|---|---|
| diameter (nm) | Thickness (nm) | (nm) | by SEM |
| 4 | ≈40 | — | 7 |
| 7 | ≈120 | — | 9 |
| 12 | ≈65 | 0.93 | 16 |
| 22 | ≈85 | 1.84 | 27 |
| 50 | ≈180 | 3.76 | 68 |
| 85 | ≈180 | 6.61 | 104 |

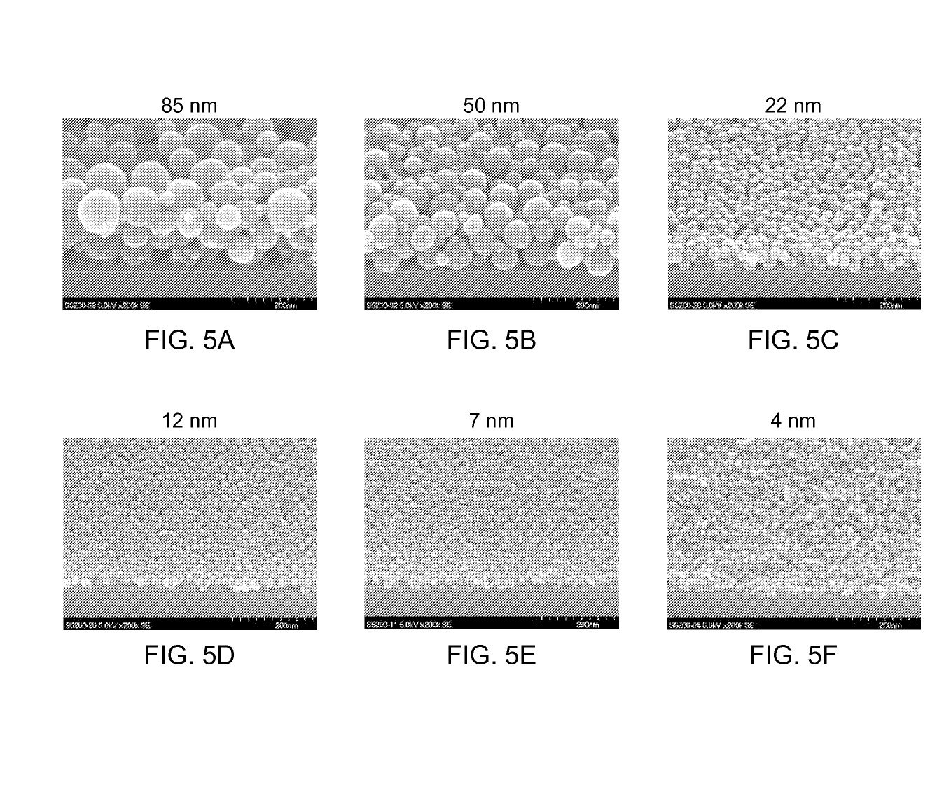

FIGS. 5A-5F show scanning electron microscopy (SEM) images of nanoparticles of nominal diameter 85 nm, 50 nm, 22 nm, 12 nm, 7 nm, and 4 nm. SEM was used to characterize the thickness of the nanoparticle coatings and the actual diameter of the nanoparticles. These values are shown in Table 3. In general, the actual diameter of the nanoparticles was slightly larger than the nominal diameter of the nanoparticles (i.e., the diameter as provided by the manufacturer). In the following examples, the stated diameter of the nanoparticles is the nominal diameter of the nanoparticles. Field emission SEM imaging and measurements were performed with an S-5200 scanning electron microscope (Hitachi High Technologies, Tokyo, Japan).

Grazing-incidence small-angle X-ray scattering (GISAXS) was used to study the morphology and organization of the nanoparticle coatings. The beamline BL03XU at the SPring-8 synchrotron at the Japan Synchrotron Radiation Research Institute was used to generate X-rays at 12.4 keV and 8.3 keV. Small-angle X-ray scattering (SAXS) patterns were detected with a charge-coupled device (CCD) camera (1344×1024 pixels, 63 μm/pixel) positioned 2330 mm from the nanoparticle coating sample. The calibration of the angular scale was performed with a collagen standard sample (d-spacing: 65.3 nm). GISAXS was performed at incident angles above the critical angle of the silicon substrate (α_{c}=0.1° at 12.4 keV).

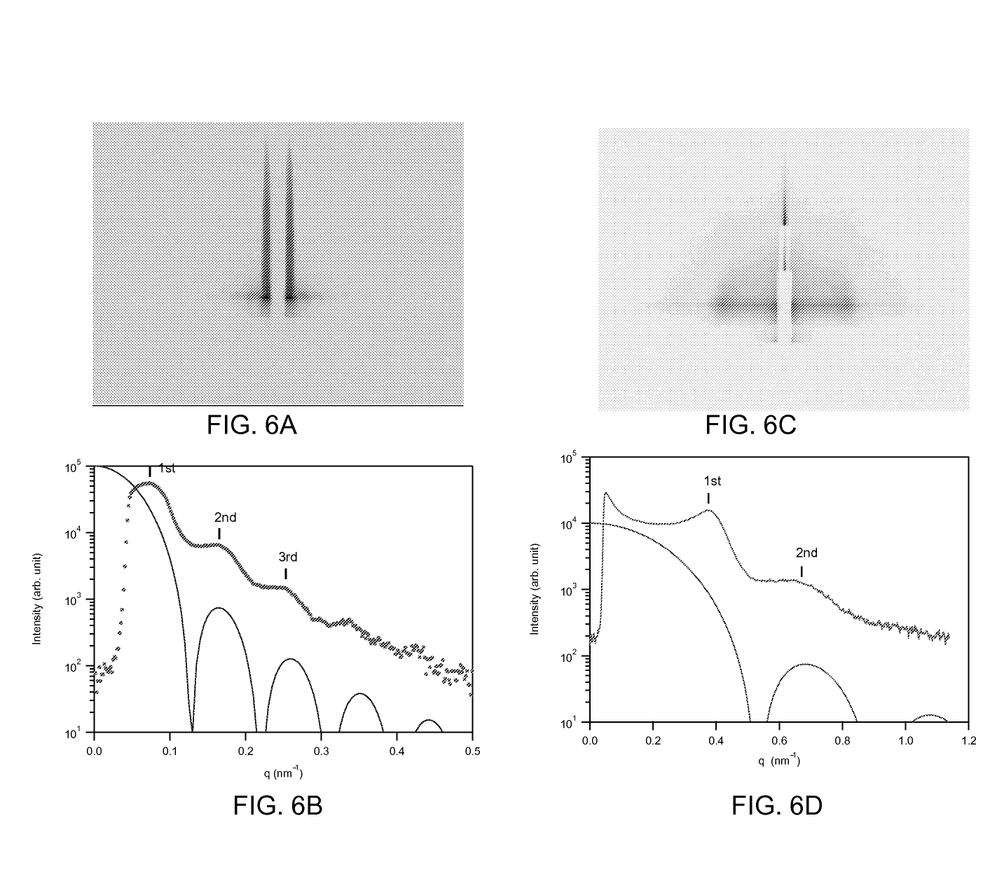

FIGS. 6A and 6B show a GISAXS image and spectrum, respectively, for a blood compatible coating of 50 nm nanoparticles. FIGS. 6C and 6D show a GISAXS image and spectrum, respectively, for a blood compatible coating of 12 nm nanoparticles. Experimentally observed GISAXS spectra 60, 62 and simulated GISAX spectra 64, 66 are shown. The GISAXS spectra present sharp in-plane Bragg peaks, indicative of highly ordered nanoparticles in the coatings. The positions of the peaks can be used to calculate the diameter of the nanoparticles in the coatings. The results of these calculations are shown in Table 4. In plane, additional diffractions are observed at higher qx values for 12 nm and larger nanoparticles, indicative of scattering by the nanoparticles.

TABLE 4
Nanoparticle diameters calculated from GISAXS spectra

| Nominal | Diameter (nm) calculated |
|---|---|
| diameter (nm) | from GISAXS |
| 85 | 114.2 |
| 50 | 71.7 |
| 22 | 29.9 |
| 12 | 17.4 |
| 7 | 10.6 |
| 4 | — |

### Example 2

### Coagulation Activity in Suspensions of Nanoparticles

The time dependent intrinsic blood coagulation activity was evaluated in suspensions of silica nanoparticles of different sizes. Flat SiO₂ glass was used as a control sample. Because FXII adsorption on the surface of the procoagulant (i.e., nanoparticles or flat glass) is a trigger of the coagulation cascade, the intrinsic coagulation activity depends on the surface area of the procoagulant. Thus, the intrinsic blood coagulation activity was also evaluated as a function of the total surface area of the silica nanoparticles in the suspensions.

To prepare nanoparticle samples for evaluation of the intrinsic coagulation activity in solution, a sample solution was formed of 10 mL of 0.1 M tris HCl, 0.6 mL of 5 N NaCl (aq)., 0.4 mL of 0.5 M CaCl₂ (aq), 0.5 mL of 2 mM phosphatidylserine (aq) (Sigma-Aldrich), 0.4 mL of 5 mM S-2238 (aq) (Chromogenix, Milan, Italy), and 0.5 mL of human plasma (Plasma Control N, Siemens Healthcare, Malvern, Pa.).

A dispersion of silica nanoparticles of the desired size (4 nm, 7 nm, 12 nm, 22 nm, 50 nm, and 85 nm diameter) was added at the desired concentration to achieve a desired total surface area of nanoparticles. DI water was added until the total volume of the sample was 18 mL. 180 μL aliquots of the sample were poured into a biologically inert MPC polymer (poly(2-methacryroyloxyethylphosphorylcholine)-coated 96-well plate (Lipidure®-Coat S-F96, NOF Corporation, Tokyo, Japan) and incubated at 37° C. for up to at least 450 minutes to enable the generation of thrombin by contact with samples. After incubation, the absorbance of each sample at 405 nm was measured in a microplate reader to quantify the amount of thrombin generated, which was used as a measure of coagulation activity.

To prepare flat glass control samples, glass cover slips were sonicated in acetone and ethanol and dried under nitrogen flow. The substrates were incubated in the sample solution (without nanoparticles) and evaluated as described above.

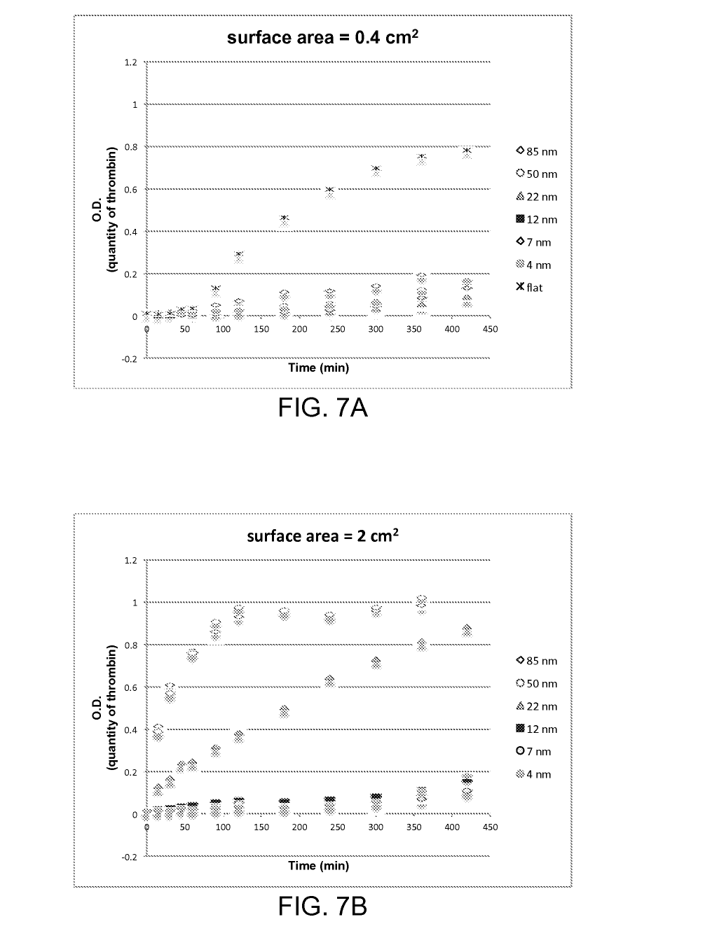

FIGS. 7A-7D show the time dependence of the intrinsic coagulation activity for nanoparticles of various sizes and for samples having 0.4 cm² total surface area of nanoparticles in the 180 μL aliquot (FIG. 7A), 2 cm² total surface area of nanoparticles (FIG. 7B), 4 cm² total surface area of nanoparticles (FIG. 7C), and 10 cm² total surface area of nanoparticles (FIG. 7D). The “flat” sample is a flat glass substrate with a surface area of 0.4 cm². The vertical axis shows the optical density (O.D.) at 405 nm after incubation, which corresponds to the quantity of thrombin generated and is indicative of the coagulation activity.

When the surface area was 0.4 cm² of nanoparticles (FIG. 7A), only flat glass activated the intrinsic blood coagulation system, while nanoparticles were almost inactive. For higher surface areas, the activation of the coagulation system became more prominent for larger nanoparticles. For the highest surface area (10 cm² of nanoparticles; FIG. 7D), all of the nanoparticles showed some activation of the intrinsic coagulation system. That is, smaller nanoparticles can inhibit the activation of the intrinsic blood coagulation to a greater degree than larger nanoparticles.

The intrinsic coagulation activity of nanoparticles with surface areas 2 cm² of nanoparticles and 4 cm² of nanoparticles after six hours (300 minutes) of incubation at 37° C. was also measured. An MPC polymer-coated well plate was used as a control due to its biologically inert properties.

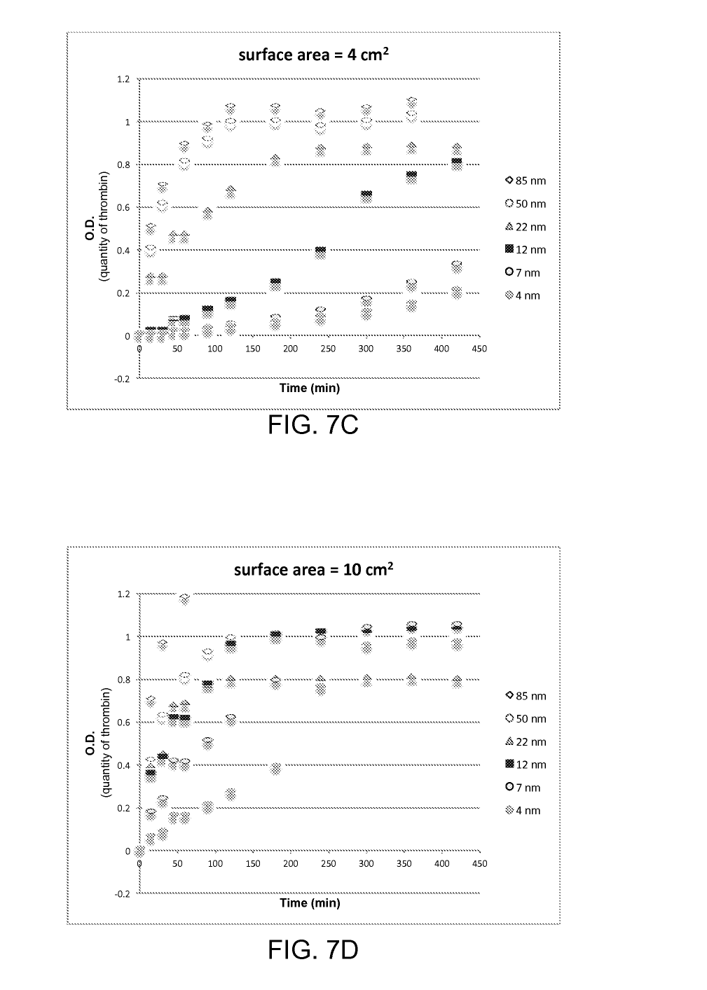

As shown in the bar graph of FIG. 8, the intrinsic coagulation activity after five hours of incubation has a clear dependence on the size of the nanoparticles, with the intrinsic coagulation activity decreasing with decreasing nanoparticle size. The vertical axis shows the O.D. at 405 nm. The intrinsic coagulation activity of the smallest nanoparticles (4 nm, 7 nm, and 12 nm) at 2 cm² surface area is generally comparable to the biologically inert MPC control sample.

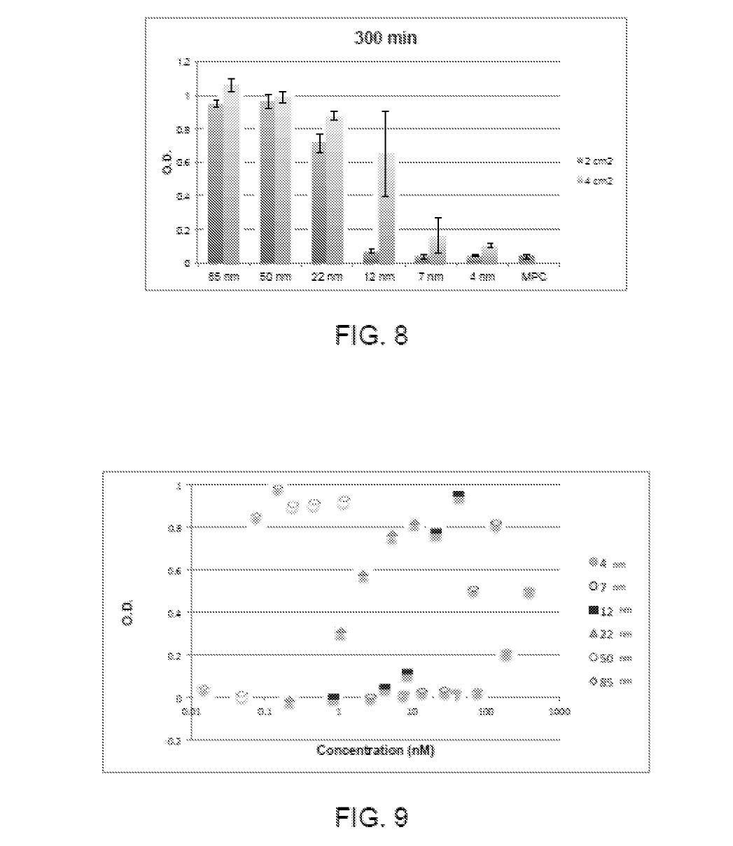

As shown in the graph of FIG. 9, the intrinsic coagulation activity after incubation for 90 minutes was measured as a function of nanoparticle size and concentration. The vertical axis shows the O.D. at 405 nm. Each nanoparticle size has a corresponding threshold concentration for the activation of coagulation activity, suggesting that the intrinsic coagulation pathway is activated only after critical quantities of FXII are adsorbed onto the pro-coagulant surface and activated to FXIIa. This threshold concentration shifts higher with decreasing nanoparticle size, suggesting that larger nanoparticles activate more FXII for a given nanoparticle concentration. That is, lower curvature (larger diameter) surfaces are more active in the coagulation system. These results agree with the results of FIGS. 6A-6D, in which flat glass was shown to be more active than even the largest tested nanoparticles.

Hydrodynamic measurements were performed with Zetasizer Nano (Malvern Instrument Ltd., Worcestershire, UK) to determine the size of the nanoparticle aggregates in the nanoparticle suspensions used in the experiments above. Table 5 below shows the average particle size for each nominal nanoparticle diameter at pH 9.0 and pH 7.4, respectively. In water of pH 9.0, silica nanoparticles are dispersed as almost single particle due to electric repulsion between particles, except for 4 nm diameter particles. That is, the average particle sizes of silica nanoparticles are almost same as the nominal diameter of the nanoparticles. In a solvent of pH=7.4, nanoparticles of all sizes aggregate. The increase in the aggregate size with increasing nominal nanoparticle diameter was not monotonic. Thus, the results above indicating the dependence of coagulation activity on nanoparticle diameter do not necessarily suggest that coagulation activity depends on the size of the nanoparticle aggregates, but rather that coagulation activity depends on the surface curvature of the features on the surface (i.e., the nanoparticles in the blood compatible coating).

TABLE 5
Average particle size of nanoparticle aggregates

| Nominal | Average particle size | Average particle size |
|---|---|---|
| diameter (nm) | at pH = 9 (nm) | at pH = 7.4 (nm) |
| 4 | 11.5 | 22.7 |
| 7 | 7.7 | 14.7 |
| 12 | 9.0 | 18.6 |
| 22 | 17.9 | 31.4 |
| 50 | 55.6 | 66.8 |
| 85 | 80.9 | 94.2 |

### Example 3

### Coagulation Activity on High Curvature Blood Compatible Coatings

The intrinsic blood coagulation activity on substrates coated with high curvature blood compatible coatings formed of nanoparticles of various sizes was characterized. Flat SiO₂ substrates and biologically inert MPC polymer substrates were used as control samples.

Blood compatible coatings of silica nanoparticles were prepared as described in Example 1 to coat both sides of a 5 mmφ cover glass with blood compatible nanoparticle coatings. Flat SiO₂ substrates were prepared as described in Example 2.

A sample solution was formed of 10 mL of 0.1 M tris HCl, 0.6 mL of 5 N NaCl (aq)., 0.4 mL of 0.5 M CaCl₂ (aq), 0.5 mL of 2 mM phosphatidylserine (aq), 0.4 mL of 5 mM S-2238 (aq), and 0.5 mL of human plasma. 5 mm×5 mm glass cover slips were coated with nanoparticles according to the approach described in Example 1 and placed into an MPC coated 96-well plate. A 180 μL aliquot of the sample was poured over each cover slip and incubated at 37° C. for up to at least 300 minutes to enable the generation of thrombin by contact with substrates. After incubation, the absorbance of each sample at 405 nm was measured in a microplate reader to quantify the amount of thrombin generated, which was used as a measure of coagulation activity.

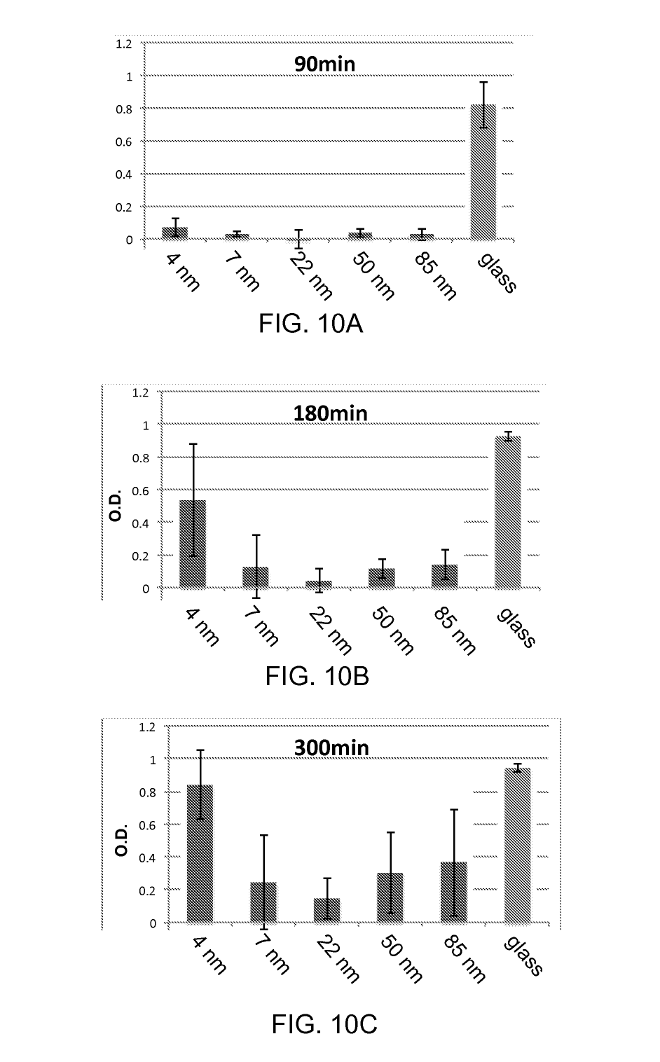

Referring to the bar graphs of FIGS. 10A-10C, the coagulation activity on nanoparticle coatings (measured as the optical density at 405 nm) was characterized after 90 minutes of incubation (FIG. 10A), 180 minutes of incubation (FIG. 10B) and 300 minutes of incubation (FIG. 10C). The vertical axis shows the O.D. at 405 nm. These results show that coagulation activity depends on the curvature of the coating (i.e., the diameter of the nanoparticles in the coating). After 90 min of incubation (FIG. 10A), flat glass activated coagulation to a nearly saturated level, while of the nanoparticle-coated surfaces barely activated the coagulation system. These data suggest that nanoparticle-coated surfaces are less active for intrinsic coagulation than a flat surface. After additional incubation, the coagulation system was gradually more activated by the nanoparticle-coated surfaces, as shown in FIGS. 10B and 10C. In general, less coagulation activity occurred on high curvature surfaces (i.e., coatings of small nanoparticles) than on flat glass. The coagulation activity decreased with decreasing nanoparticle size until the coating of 22 nm diameter nanoparticles, which demonstrated the least coagulation activity of the coatings studied. Still smaller nanoparticles showed increased coagulation activity, but the activity was smaller than the coagulation activity on flat glass.

The results for coagulation activity on surfaces are somewhat different from the results for coagulation activity in suspensions of nanoparticles (Example 2). In particular, coagulation activity in suspensions of nanoparticles decreased continuously with decreasing nanoparticle size, while a local minimum in coagulation activity was observed for the 22 nm diameter nanoparticle coating. Nanoparticles in blood compatible coatings are densely packed (Example 1), and thus the distance between nanoparticles in a coating is very short. For coatings formed of very small nanoparticles, such as 4 nm diameter nanoparticles, the distance between nanoparticles can be smaller than the size of the proteins involved in the coagulation activity (e.g., FXII). Without being bound by theory, it is believed that proteins may recognize coatings formed of very small nanoparticles as essentially flat surfaces, and hence the coagulation activity on such nanoparticle coatings can be increased.

### Example 4

### Platelet Adhesion on High Curvature Blood Compatible Coatings

To characterize the ability of nanoparticle coatings to prevent platelet adhesion, substrates coated with silica nanoparticle coatings of various sizes were incubated in the presence of platelets. Flat SiO₂ substrates and biologically inert MPC polymer substrates were used as control samples. The number and morphology of the platelets that adsorbed on each substrate were characterized.

Substrates with silica nanoparticle coatings and flat SiO₂ substrates were prepared on 1 cm² silicon wafer as described in Example 3. To prepare a flat MPC polymer coated surface, a 1 cm² Si wafer was sonicated in acetone and ethanol and dried under nitrogen flow. The substrate was then treated by oxygen plasma for 10 minutes. 0.5 wt. % MPC polymer (Lipidure®-CM5206, NOF Corporation, Tokyo, Japan) in ethanol was spin coated onto the substrate (3000 rpm, 160 seconds) and dried under ambient conditions.

Citrated pooled whole blood (Bioreclamation Inc., Westbury, N.Y.) was centrifuged at 300 G for 10 minutes, and the supernatant was collected as platelet rich plasma (PRP). Substrates were incubated with 1 mL PRP on an MPC polymer coated 24-well plate (Lipidure®-Coat S-F24, NOF Corporation, Tokyo, Japan) at 37° C. under the condition of 5% CO₂ for three hours. The substrates were rinsed with 0.1 M phosphate buffer and fixed following general procedure. The adsorbed platelets on each substrate were observed by optical microscopy and the number of platelets per 100 μm×100 μm area were counted.

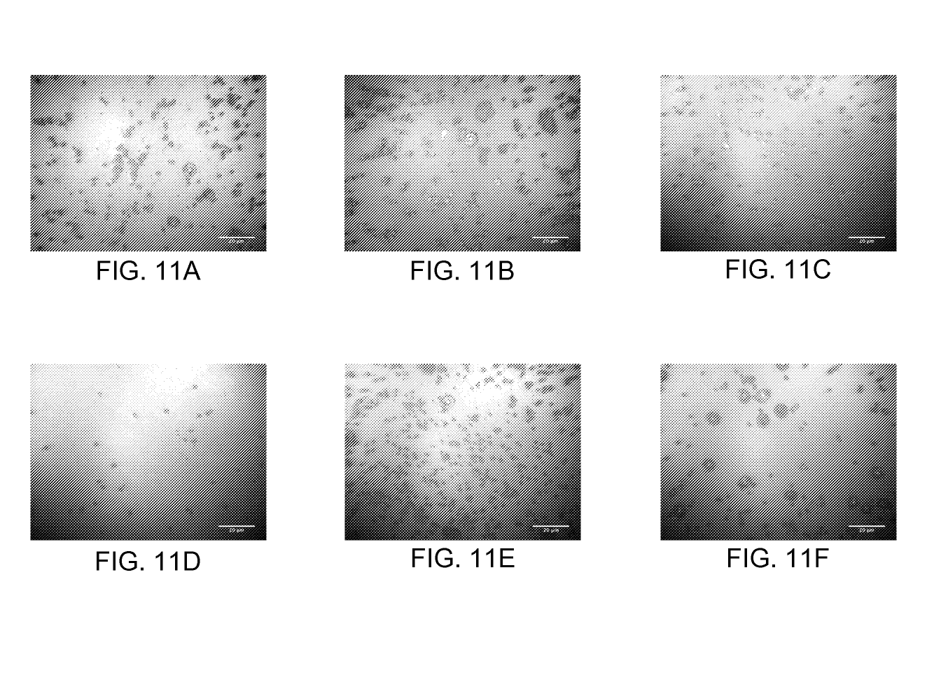

FIGS. 11A-11F show optical microscopy images (scale bar: 20 μm) of platelets adhered to surfaces with coatings of nanoparticles with 4 nm diameter (FIG. 11A), 12 nm diameter (FIG. 11B), 22 nm diameter (FIG. 11C), and 85 nm diameter (FIG. 11D). Platelets adhered to a flat SiO₂ substrate (FIG. 11E) and a flat MPC polymer coating (FIG. 11F) are also shown. The coatings of 22 nm (FIG. 11C), 50 nm, and 85 nm (FIG. 11D) diameter nanoparticles adsorbed fewer platelets than the coatings of smaller nanoparticles. In addition, the platelets adsorbed on the larger nanoparticles had a rounder morphology than those adsorbed on the smaller nanoparticles. Round platelets are the least activated form of platelets. Thus, these optical microscopy images indicate that the platelets adsorbed on large nanoparticles are not highly activated, suggesting that blood clots may not form on coatings of large nanoparticles.

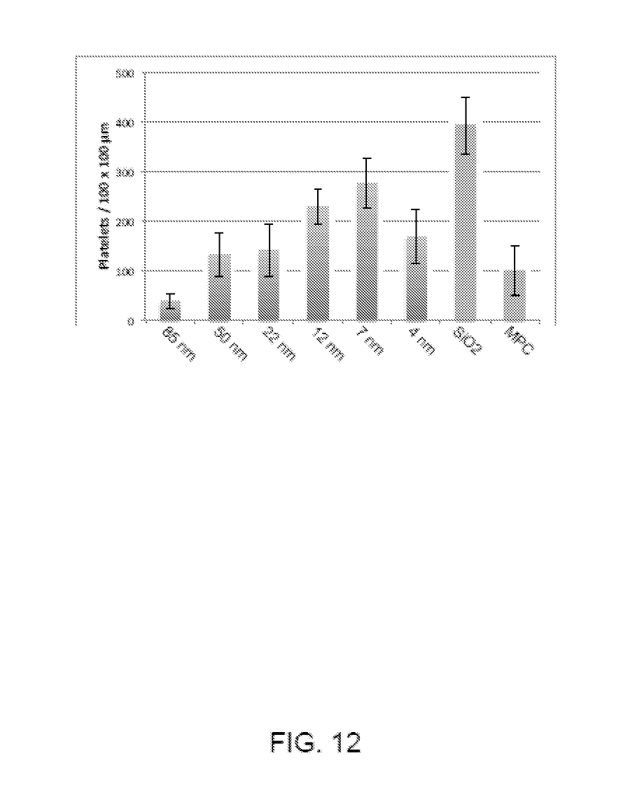

FIG. 12 shows a plot of the number of platelets adsorbed in 100 μm×100 μm area for each nanoparticle coating and for the two control samples. The number of platelets adsorbed to the flat SiO₂ sample was greater than the number of platelets adsorbed to any of the nanoparticle coatings, indicating that silica nanoparticles of 85 nm diameter or less prevent platelet adhesion more effectively than flat SiO2. The size of the nanoparticles does affect platelet adhesion: the coating of 7 nm diameter nanoparticles had the most adsorbed platelets and the coating of 85 nm diameter nanoparticles had the fewest adsorbed platelets. Moreover, the 85 nm diameter nanoparticle coating had fewer adsorbed platelets than the MPC polymer, which is a highly blood compatible material.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of limiting blood coagulation and platelet adhesion to a surface of a medical device, the method comprising:
flowing blood over the surface of the medical device, wherein the surface of the medical device is a rough surface that comprises a plurality of three-dimensionally curved features each having a radius of curvature of less than about 50 nm, and
wherein the surface of the medical device comprises a sufficient concentration of features per unit area to limit blood coagulation activity on the medical device and to limit the number of platelets that adhere to the surface when the medical device is exposed to blood.

2. The method of claim 1, wherein the medical device is an implantable medical device and the method is carried out in vivo.

3. The method of claim 1, wherein the medical device is configured for exposure to blood outside of the body of a patient and the method is carried out ex vivo.

4. The method of claim 1, wherein the surface of the medical device comprises a coating, and wherein the coating comprises the features.

5. The method of claim 4, wherein the features comprise nanoparticles, and wherein a fill rate of the nanoparticles in the coating is at least about 50%.

6. The method of claim 4, wherein the features are nanoparticles having a diameter of less than about 100 nm.

7. The method of claim 1, wherein an RMS roughness of the surface of the medical device is less than about 10 nm.

8. A method of making a blood compatible article, the method comprising:
providing a substrate; and
forming a rough surface on the substrate, wherein the rough surface comprises a plurality of three-dimensionally curved features each having a radius of curvature of less than about 50 nm,
wherein the surface comprises a sufficient concentration of features per unit area to limit blood coagulation activity on the substrate and to limit the number of platelets that adhere to the surface when the substrate is exposed to blood.

9. The method of claim 8, wherein the three-dimensionally curved features are substantially hemispherical.

10. The method of claim 8, wherein the rough surface comprises a coating on the substrate, and wherein the coating comprises the features.

11. The method of claim 10, wherein the features comprise nanoparticles, and wherein a fill rate of the nanoparticles in the coating is at least about 50%.

12. The method of claim 8, wherein the features are nanoparticles having a diameter of less than about 100 nm.

13. The method of claim 12, wherein forming the coating includes one or more of spin coating the nanoparticles onto the surface of the substrate, dip coating the nanoparticles onto the surface, spray coating the nanoparticles onto the surface, precipitating the nanoparticles onto the surface, or depositing the nanoparticles by flame spray pyrolysis.

14. The method of claim 8, wherein forming the coating includes forming the features by nano-imprinting on the substrate.

15. The method of claim 8, wherein the substrate is a medical device or a part of a medical device.

16. The method of claim 15, wherein the medical device is an implantable medical device.

17. The method of claim 15, wherein the medical device is configured for exposure to blood outside of the body of a patient.

18. The method of claim 8, wherein an RMS roughness of the surface is less than about 10 nm.

19. A blood compatible article comprising:
a substrate having a rough surface, wherein the rough surface comprises a plurality of three-dimensionally curved features each having a radius of curvature of less than about 50 nm,
wherein the surface comprises a sufficient concentration of features per unit area to limit blood coagulation activity on the substrate and to limit the number of platelets that adhere to the surface when the substrate is exposed to blood.

20. The blood compatible article of claim 19, wherein the features are substantially hemispherical.

21. The blood compatible article of claim 19, wherein the rough surface comprises a coating on the substrate, and wherein the coating comprises the features.

22. The blood compatible article of claim 21, wherein the features comprise nanoparticles, and wherein a fill rate of the nanoparticles in the coating is at least about 50%.

23. The blood compatible coating of claim 22, wherein the fill rate of the nanoparticles in the coating is at least about 70%.

24. The blood compatible article of claim 19, wherein the features are nanoparticles having a diameter of less than about 100 nm.

25. The blood compatible article of claim 19, wherein the substrate is a medical device or a part of a medical device.

26. The blood compatible article of claim 19, wherein the concentration of the features limits the adsorption onto the substrate of one or more of Factor XII or a protein associated with coagulation.

27. The blood compatible article of claim 19, wherein the limited coagulation activity inhibits formation of a fibrin clot at the surface of the substrate.

28. The blood compatible article of claim 19, wherein the concentration of the features limits the activation of platelets adsorbed on the substrate.

29. The blood compatible article of claim 19, wherein an RMS roughness of the surface is less than about 10 nm.
